(19) United States
(12) Patent Application Publication
(10) Pub. No.: US 2024/0024586 A1
(43) Pub. Date: Jan. 25, 2024

(54) MEDICAL SYRINGE

(71) Applicant: TRENTA2 S.R.L., Bozen (IT)

(72) Inventor: Dietrich GALLMETZER, Terlan (IT)

(73) Assignee: TRENTA2 S.R.L., Bozen (IT)

(21) Appl. No.: 18/265,693

(22) PCT Filed: Dec. 6, 2021
(86) PCT No.: PCT/EP2021/084454

§ 371 Date: Jun. 7, 2023

(30) Foreign Application Priority Data
Dec. 7, 2020 (EP) 20212250.3

(51) Int. Cl. A61M 5/32

(52) U.S. Cl. CPC A61M 5/322; A61M 5/3202

(57) ABSTRACT

A medical syringe with a hollow body forming the syringe housing and intended for receiving a medical active ingredient, on the distal end of which a connecting element is integrally formed, on which a needle holder for a hollow needle intended for injecting the active ingredient can be attached, is to be designed for the lowest possible loss of material with regard to the active ingredient to be dispensed. For this purpose, the connecting element has an inner channel provided for connection to the needle holder, which has an inner cross-section that widens continuously towards the distal end.

### FIELD OF INVENTION

The invention relates to a medical syringe, in particular with needle protection, with a hollow body forming the syringe housing and intended to receive a medical active ingredient.

### BACKGROUND

Medical syringes are used in a wide variety of applications, particularly therapeutic ones. They usually comprise a piston that can be moved in the interior of a syringe body via an actuating plunger.

To avoid or reduce the risk of contamination or injury after the use of medical syringes, and in particular to avoid multiple use of syringe needles by different users, syringes with so-called retraction systems for the syringe needle are increasingly being used. In such syringes, in particular medical syringes, also referred to as “syringes with (passive) needle protection”, the syringe needle is retracted into the syringe body after delivery of the active ingredient held in the syringe and is completely enclosed by the latter. Access to the syringe and thus a risk of injury, or even the risk of multiple use of the same needle, can thus be largely avoided.

Such syringes with passive needle protection are known, for example, from EP 1 284 769 B1, EP 0 720 491 B1, EP 0 680 347 B1 or EP 1 764 127 B1.

The needle of a medical syringe is usually held in a needle holder which is suitably connected to the syringe housing, in particular screwed or clipped onto the syringe. In the case of medical syringes in general, and thus also syringes with needle protection of the type mentioned, there is a need to deliver the active ingredient contained therein as completely as possible during use of the syringe and to minimize as far as possible the retention of unused residual amounts of the active ingredient within the syringe.

### BRIEF SUMMARY OF INVENTION

It is now an object of the invention to specify a syringe with needle protection of the type mentioned above, in which this need is taken into account even with a subsequently attached needle holder.

This task is solved according to the invention in that the connecting element has an inner channel provided for connection to the needle holder, which has an inner cross-section that widens continuously towards the distal end.

Advantageous embodiments of the invention are the subject of the subclaims and/or the following figure description.

The invention is based on the consideration that, particularly in the case of multi-component systems in which the needle holder is initially manufactured as a separate component and is only subsequently mounted on the syringe barrel, leaks could occur in the area of the connection between the needle holder and the syringe barrel, resulting in leakage. In order to counteract this and to ensure a particularly high level of system tightness even with this design, and thus to prevent the unintentional release of active ingredient due to leaks, the connection area in particular should be designed for a particularly high level of tightness. In order to make this possible, the use of a conical internal connection is now envisaged for the active substance-side connection of the needle holder and syringe housing. The internal channel provided for this purpose in the connection area of the syringe housing has a widening cross-section in the form of a cone. This means that the corresponding component on the needle holder can be inserted into this cone so that the desired tightness of the connection can be ensured, particularly if the contact pressure is sufficient.

Advantageously, the inner channel is designed in the form of a cone widening towards the distal end with a cone angle of about 2°.

In advantageous further development or in an embodiment regarded as independently inventive, the medical syringe is equipped with an RFID chip, for example for the purpose of setting up digitalized logistics, an automatable transport system for syringes, for quality or product control and/or for other recording or monitoring purposes. This advantageously has individualized, machine-readable coding. Preferably the RFID chip is arranged on a grip plate formed on the proximal end of the hollow body forming the syringe housing.

In a further alternative or additionally advantageous embodiment or also in an embodiment considered to be independently inventive, the medical syringe comprises an actuating plunger, on the shaft of which a number of stop and latching elements are arranged, preferably designed as adjusting rings running around the shaft.

In a particularly preferred embodiment, the syringe barrel is manufactured as a plastic part, preferably from cyclo-olefin polymer (COP) or cyclo-olefin copolymer (COC), with or without a barrier layer. This material is characterized by high break resistance and glass-like transparency. It also does not release any alkali ions, so that the risk of a pH shift in the active ingredient held in reserve is excluded. Thus, according to the invention, this material is considered and used as suitable even for primary packaging of even demanding drugs, especially for sensitive biotechnologically produced active ingredients. In addition, this material is suitable for production by injection molding and thus for particularly precise dimensioning.

### BRIEF DESCRIPTION OF THE DRAWINGS

An embodiment of the invention is explained in more detail with reference to a drawing. Therein show:

FIG. 1A medical syringe in longitudinal section in two cutting planes,

FIG. 2 a hollow body forming the syringe housing of the syringe according to FIG. 1 in longitudinal section and in lateral view,

FIG. 3 a needle holder of the syringe according to FIG. 1 in longitudinal section,

FIG. 4 a side and perspective view of an actuating plunger of the syringe according to FIG. 1,

FIG. 5 the actuating plunger according to FIG. 4 with attached piston,

FIG. 6 a protective cap of the syringe according to FIG. 1 in perspective view and in longitudinal section,

FIGS. 7, 8 the syringe according to FIG. 1 in perspective view with or without protective cap,

FIG. 9 the syringe according to FIG. 1 in an actuation position in longitudinal section,

FIG. 10 the hollow body according to FIG. 2 in perspective view,

FIGS. 11, 12 the syringe shown in FIG. 1 in different actuation positions in longitudinal section,

FIGS. 13, 14 a perspective view of the hollow body as shown in FIG. 2,

FIG. 15 a standard syringe equipped with RFID chip,

FIGS. 16, 17 each an exploded view of a medical syringe with a passive retraction mechanism, and

FIG. 18 a hollow body of the syringe according to FIGS. 16, 17.

Identical parts are provided with the same reference signs in all figures. The embodiments have a plurality of features or feature groups regarded as independently inventive. In accordance with the invention, these can each be provided individually and independently of one another or also in any combination with one another.

### DETAILED DESCRIPTION OF THE DRAWINGS

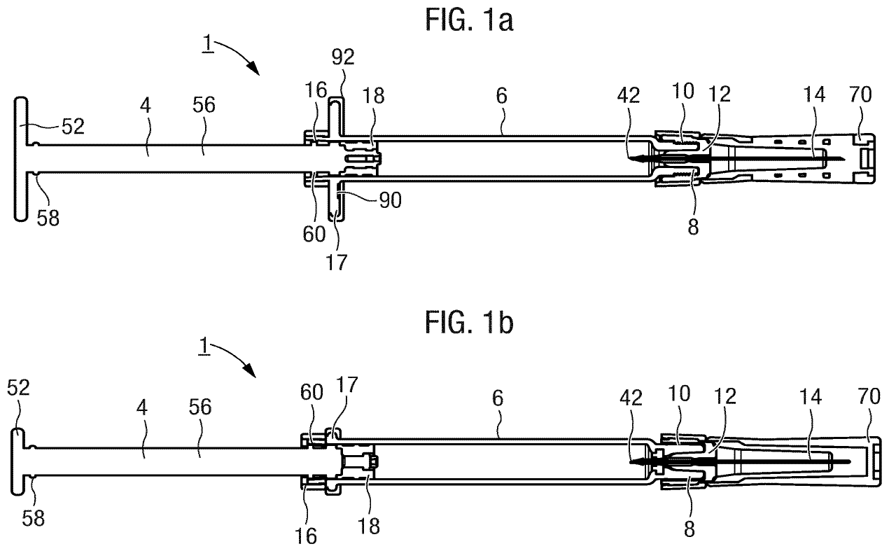

The medical syringe 1 with needle retraction, which is shown in its entirety in longitudinal section in FIG. 1, comprises an actuating plunger 4 and a hollow body 6, which is of cylindrical or tubular design in accordance with a conventional design, forms the syringe housing, and is provided for receiving the medical active ingredient. A connecting element 10 is formed on the front or distal end 8 of the hollow body 6, to which a needle holder 12 for a hollow needle 14 provided for injection of the active ingredient can be attached. The rear or proximal end 16 of the hollow body 6 forming the syringe housing, on which a counter or grip plate 17 is integrally formed in a manner known per se, can, on the other hand, be closed by a piston or stopper 18 whose outer dimensions fit exactly to the inner contour of the hollow body 6 and which can be displaced within the hollow body 6. The medical syringe 1 could in principle be designed as a syringe 1 pre-filled with the active ingredient or, alternatively, it could also be used in such a way that the syringe 1 is made ready for use when empty and is filled with the active ingredient by drawing it on. With regard to the detailed design features described below, both types of use are considered to be equivalent and can be used as alternatives to one another.

In view of the concept that the syringe should be able to be used well and reliably in modern therapies and the sensitive, high-priced and, under certain circumstances, toxic active ingredients that may be used, the syringe and, in particular, the hollow body 6 forming the syringe housing are designed to be particularly leakproof so that, especially when used as a prefilled syringe, a certain shelf life of the active ingredient stored therein can also be achieved. For this purpose, on the one hand, the material for the syringe housing or the hollow body 6 forming it is suitably selected in a manner considered to be independently inventive, so that even high demands on the reliable temporary storage of the active medical ingredient are taken into account with a particularly high level of safety in the handling of the components. The injection body 6, which is designed as a cylindrical hollow body, can be made of PP or cyclo-olefin copolymer (COC), with or without a barrier layer, but in the embodiment considered to be in line with the invention it is particularly preferably made of the high-performance plastic cyclo-olefin polymer (COP). This material is characterized by high break strength and glass-like transparency. It also does not release any alkali ions, so that the risk of a pH value shift in the active ingredient being held is excluded. The syringe barrel 6 is preferably manufactured by injection molding, whereby, among other things, it is shaped in such a way that possible dead volumes in the interior are kept particularly low.

On the other hand, the hollow body 6 is also suitably designed for the intended high tightness and thus protection against unintentional loss of material by the geometric design and shaping of its components. This is taken into account in the area of the distal end 8 in particular by the design of the geometry of the connecting element 10 and the component connection formed by this together with the needle holder 12, which is considered to be independently inventive.

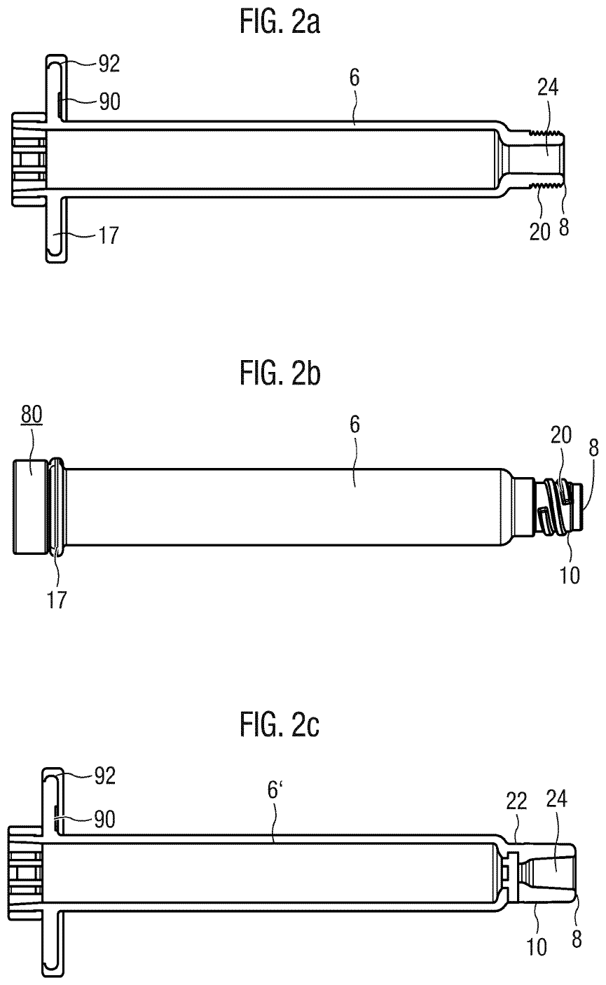

The needle holder 12 can be attached to the hollow body 6 forming the syringe housing or to the syringe cone, or alternatively it can be screwed on by means of a thread, for example a Luer thread. Both variants are regarded as equivalent alternatives, whereby the relevant design of the connection is regarded as interchangeable and freely selectable with regard to other boundary conditions, for example the intended subsequent handling. Accordingly, in the embodiment according to FIG. 2, FIG. 2a shows in longitudinal section and FIG. 2b in lateral view a hollow body 6 forming the syringe housing, the connection element 10 of which is provided with an external thread 20 intended for the production of a screw connection. As shown in FIG. 2b, this is preferably designed as a Luer thread, but can also be designed as a fine thread, as shown in FIG. 2a. In contrast, a longitudinal section of a hollow body 6′ forming the syringe housing is shown in FIG. 2c, the connecting element 10 of which is provided with a holding contour having a detent edge 22, for example in the form of an external hexagon, for producing a clip, push-on or bounce-on connection.

Both variants have in common the design, which is regarded as independently inventive, that the connecting element 10 has in each case an inner channel 24 which is provided for connection to the needle holder 12 and has an inner cross section which widens continuously outwards, i.e. towards the distal end 8. In the embodiment example, the inner channel 24 is designed in each case in the manner of a cone widening towards the free end with an at least approximately circular cross-section. The angle of the cone is suitably selected with regard to the intended sealing effect, which is explained in more detail below; with regard to the preferably selected material pairing of the hollow body 6 and the needle holder 12, the angle of the cone is preferably about 2°.

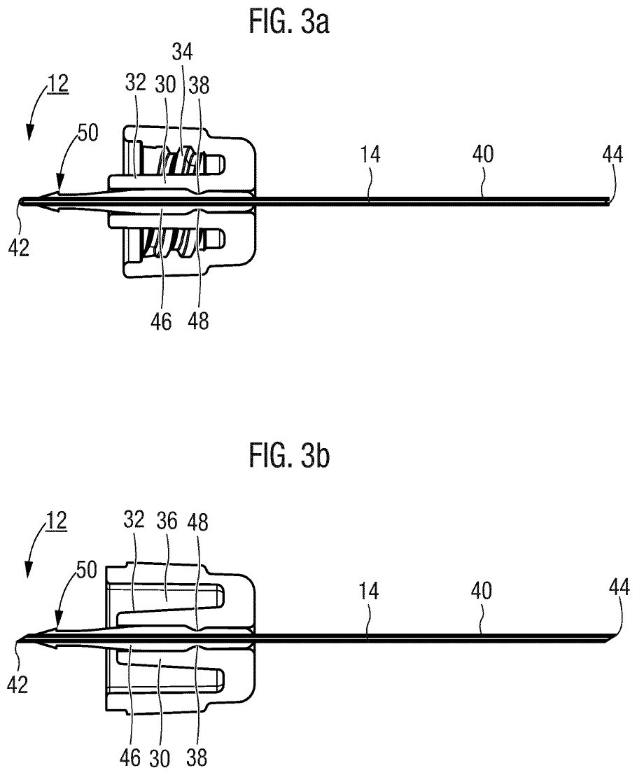

The needle holder 12 carrying the hollow needle 14 is shown in detail in longitudinal section in FIG. 3, namely in FIG. 3a in a variant provided for the screw connection and in FIG. 3b for the clip or plug connection. It has in each case a bearing sleeve 30 for the hollow needle 14. This has a conically designed contact surface 32 adapted in its dimensioning, i.e. with regard to its cross-sectional area, its diameter and its cone angle, to the inner channel 24 of the connecting element 10 and insertable therein. The needle holder 12 and/or its plug 30 can be of single-component or also multi-component design, in particular of two-component design. By means of the aforementioned design of the connection system, a particularly high sealing effect can be achieved with the targeted use of the conical design of the connection surfaces, with which the inner channel 24 is closed by the needle holder 12. The actual fixing to the connecting element 10 is then carried out in the variant of the screw connection shown in FIG. 3a by means of an internal thread 34 integrated in the needle holder 12 and adapted to the external thread 20 and thus also designed as a Luer thread or fine thread, and in the variant of the clip or plug connection shown in FIG. 3b by means of an internal contour adapted to the holding contour, in the embodiment example an internal hexagon 36.

The design of the hollow needle 14 and its fixation in the bearing sleeve 30 essentially correspond to the design of the system described in EP 3 738 629 A1. Analogously, the bearing sleeve 30 is also provided here with a fixing lip 38 running around. The hollow needle 14 comprises a needle tube 40 made of metal, which forms a needle point 42, 44 at each of its two ends. The material for the needle tube 40 is preferably selected with regard to common requirements for medical applications, with particular preference being given to a stainless material that can also be used for standard needles, in particular stainless steel. In its central length region, the needle tube 40 is sheathed and surrounded or injection-molded by a plastic sheath 46. The material for the plastic jacket 46 is preferably a polyamide (PA12), most preferably the one commercially available under the designation Vestamid Care ML 17. In a particularly preferred embodiment, the plastic coating 46 is sprayed onto the needle tube 40 after the latter has been subjected to a plasma pretreatment in the manner of a surface activation. In this way, particularly good adhesion of the plastic forming the plastic jacket 46 to the needle 40 can be achieved.

A retaining groove 48 is formed in the plastic jacket 46, which serves to temporarily fix the needle 14 in the bearing sleeve 30 of the needle holder 12. In this case, the fixing lip 38 of the bearing sleeve 30 engages in the holding groove 48 when the needle 14 is mounted and properly pushed into the bearing sleeve 30 and fixes it in the longitudinal direction. The dimensions of the retaining groove 48 and the fixing lip 38 are selected such that, taking into account the deformability of the material of the plastic sheath 46, the overmolded needle 14 can be pushed into the bearing sleeve 30 and clipped or engaged therein. Furthermore, the plastic sheath 46 is designed in its end region facing the needle tip 42 on the inside to form a latching edge 50 or a number of latching elements, for example latching hooks or the like.

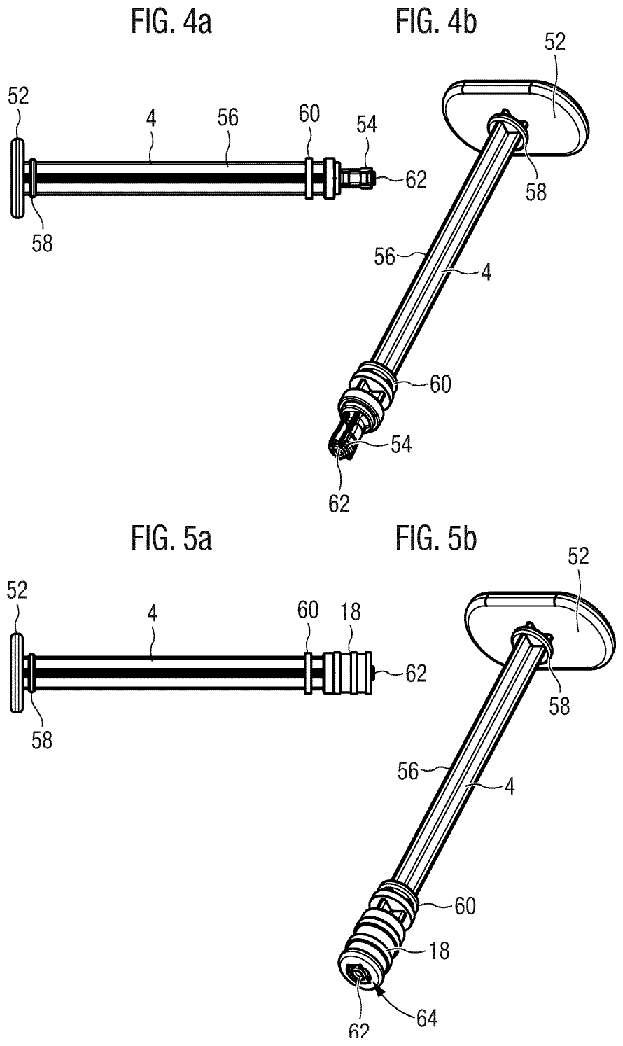

The drive or actuating plunger 4 shown in FIG. 4 in plan view and perspective alone and in FIG. 5 with attached piston 18 comprises a shaft 56 provided on one side at the end with a pusher plate 52 and on the other side at its end opposite the pusher plate 52 with a gripping yoke 54 provided for receiving the hollow needle 14. In an embodiment considered to be independently inventive, a number of stop and detent elements are also arranged on the shank 56, which in the embodiment example are designed as adjusting rings 58, 60, namely a first adjusting ring 58 arranged proximally in the region of the shank 56 adjacent to the pusher plate 52 and a second adjusting ring 60 arranged distally in the region of the shank 56 adjacent to the gripping bow 54. The design and operation of these stop and detent elements will be explained in more detail below.

The medical syringe 1 is provided with a needle protection in the form of a retraction system by the components and parts mentioned. The purpose of this is that after the syringe 1 has been used, i.e. after the active ingredient contained in the hollow body 6 forming the syringe housing has been dispensed via the needle 14, the latter is drawn into the syringe housing in such a way that it is completely enclosed by the syringe housing. This is intended to keep unintended contact with the used needle 14, for example by auxiliary or nursing personnel, and thus the risk of injury and contamination particularly low or, if possible, completely excluded. For this purpose, the medical syringe 1 is provided with a retraction system essentially corresponding to the design of the system described in EP 3 738 629 A1.

For this purpose, the gripping yoke 54 is provided centrally with a needle bearing 62 which forms a receiving hole for the needle 14 and into which the needle point 42 on the inside can be inserted and latched by means of the latching edge 50 or other suitable latching means. In this case, the needle bearing 62 is provided on the inside with an associated circumferential latching lip, analogous to the bearing sleeve 30 described above, which engages in the holding groove formed by the latching edge 50 when the needle tip 42 is inserted into the needle bearing 62 and thus fixes the needle 14 in the longitudinal direction. The dimensions of the retaining groove and of the latching lip in the needle bearing 62 associated therewith are selected in such a way that, taking into account the malleability of the material of the plastic sheath 46 and/or any adhesive force due to the material pairing of the material of the plastic sheath 46 and the material of the needle bearing 62 surrounding it, the retaining or breakaway force of the needle 14 thus latched in the longitudinal direction is greater than the corresponding retaining or breakaway force of the fixing lip 38 in the bearing sleeve 30, so that during a retraction movement of the piston 18 into the interior of the hollow body 6,6′, the needle 14 is retracted into the interior of the hollow body 6, 6′. The gripping yoke 54 is designed as a plastic part and, in the design example, with regard to the required holding forces and the mechanical loads expected during specific use, but also with regard to approval-related requirements, consists of the polypropylene available under the designation “Bormed™” (HD810MO, ISO 10993 Information (Biocompatibility)) or, alternatively, of other polyolefins suitable for pharmaceutical use with corresponding suitability.

Also analogous to the system described in EP 3 738 629 A1, the design of the gripper bow 54 in combination with the piston 18 surrounding it creates a free space 64 inside the piston 18 which, in the final phase of dispensing the active ingredient, when the needle tip 42 has already penetrated the needle bearing 62 and is thus no longer readily accessible to the active ingredient, allows the active ingredient to flow into the needle tube 40 via the needle tip 42 in the manner of a bypass. In this case, the inflow can occur on both sides of the gripping yoke 54 into the free space 64. The stopper 18, which thus functions as a piston jacket, is advantageously made of conventional rubber, particularly preferably of the material available from Kreiburg under the designation TM4RST (MC/RS Series), taking into account approval-related requirements. The stopper 18 is shaped in such a way that, when the gripper 54 is inserted, it leaves free inflow channels for the active ingredient on both sides of the gripper 54, so that the bypass for the desired zero-volume discharge is formed in the sense of avoiding or minimizing the dead volume.

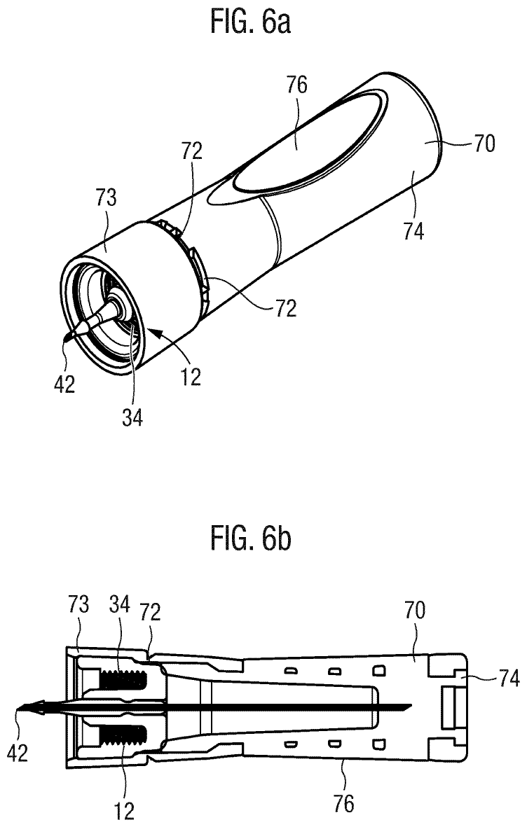

To prevent injuries or the like when handling the medical syringe 1, it is further provided with a removable protective cap 70 for the needle 14, which is removed before the syringe 1 is used. This protective cap 70 shown in FIG. 6 is in a very particularly preferred design as a so-called tamper-evident closure in the manner of a disposable closure. This design as a disposable or tamper-evident closure permits problem-free and reliable identification of whether or not the syringe 1 has already been used for liquid transfer, and thus facilitates the assignment of whether or not the syringe 1 still meets the requirement for sterility or compliance with the sterile barrier, for example, or whether or not—when used as a prefilled syringe—the active ingredient supply held therein has already been “broken into” or not. As can be seen from the perspective view in FIG. 6a and the longitudinal section in FIG. 6b, the protective cap 70 is connected to a tear-off or retaining ring 73 by a number of connecting webs 72, i.e., for example, molded or cast on. The tear-off or securing ring 73 can be clipped onto the needle holder 12 in a latching manner. To remove the protective cap 70, it must thus be broken off with from the tear-off or securing ring 73, whereby the connecting webs 72 are severed in a manner that cannot be repaired. In the embodiment shown, the protective cap 70 forming the tamper-evident closure has a two-component design and comprises the actual cap housing 74 (preferably formed from PP) as well as a control panel 76 (preferably made from TPE).

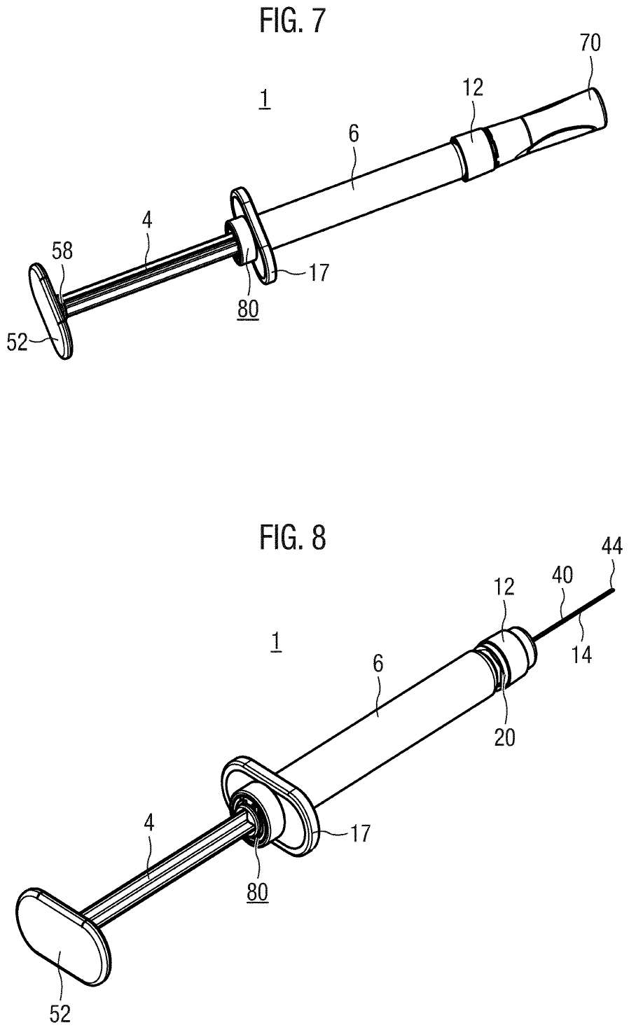

The medical syringe 1 in its entirety, i.e. with the actuating plunger 4, the hollow body 6, 6′ forming the syringe housing and the attached tamper-evident closure, prior to its first use is shown in perspective view in FIG. 7 and, as already explained, in longitudinal section in FIG. 1. When using and handling the above-mentioned components, the following procedure is basically provided for:

The first step is to remove the protective cap 70 by breaking it off. Thereafter, the syringe 1 is ready for use, as shown in FIG. 8. The syringe 1 could in principle be used with the hollow body 6 pre-filled, i.e. as a pre-filled syringe. In this case, the system is already ready to dispense the active ingredient in the state shown in FIG. 8.

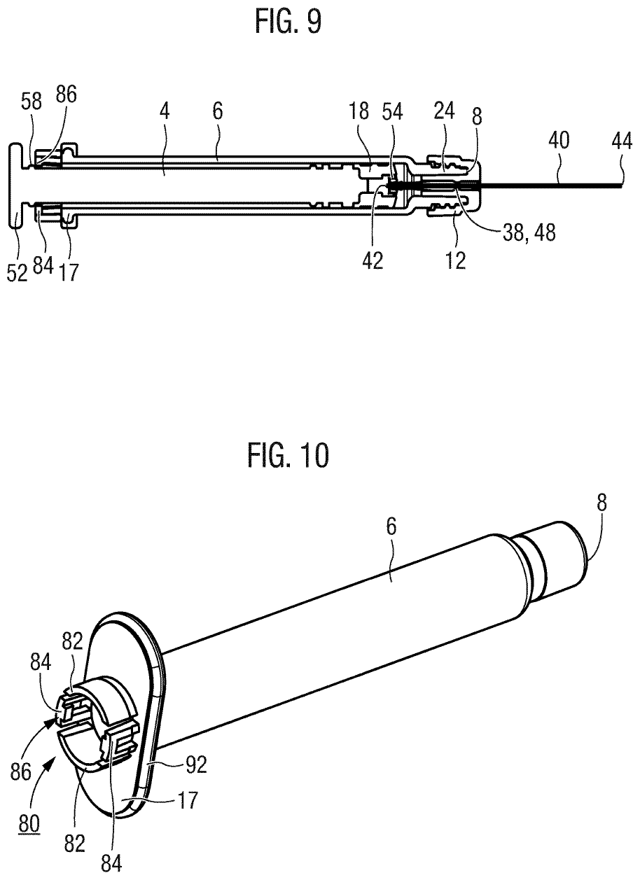

In the embodiment shown, however, it is intended that the syringe 1 be made ready for use when empty and filled with the active ingredient by drawing it on. For this purpose, the operator first presses the actuating plunger 4 so that the stopper 18 moves inside the hollow body 6 towards its distal end 8. As soon as a predetermined end position is reached in which the stopper 18 is still sufficiently far away from the needle tip 42 so that the detent edge 50 cannot penetrate the needle bearing 62 in the gripper bow 54, this movement is stopped by a stop. The syringe 1 in this position is shown in longitudinal section in FIG. 9. The stop is formed here in an embodiment considered independently inventive by the interaction of the proximal setting ring 58 with a detent receptacle 80 formed on the outside of the grip plate 17 of the hollow body 6.

The detent receptacle 80, the exact design of which according to the embodiment example is clearly visible in the perspective view of the handle plate 17 of the hollow body 6 according to FIG. 10, comprises a number of ring segments 82 which form a guide contour in their entirety, are integrally formed on the handle plate 17, and form a not completely continuous circumferential contour with the formation of gaps. Outwardly bendable latching hooks 84 are arranged in each of the gaps, each of which forms a stop surface for the proximal adjusting ring 58 on its upper side 86.

When the stopper 18 is pushed into the interior of the hollow body 6, this abutment of the proximal setting ring 58 against the upper side 86 of the latching hook 84 is palpable for the user or operator, so that he can stop the insertion movement in the position shown in FIG. 9. In what is considered to be an inventive embodiment, the positioning of the setting ring 58 on the shaft 56 is chosen in such a way that the stopper 18 is still sufficiently far away from the needle tip 42 so that the latching edge 50 cannot penetrate the needle bearing 62 in the gripper bow 54. This ensures that an unintentional “too early” release of the retraction mechanism can be avoided. In this position, the exposed distal needle tip 44 of the needle 14 can now be inserted into an external reservoir of the active ingredient, for example a vial or an active ingredient container. Subsequently, the stopper 18 is retracted in the hollow body 6 by means of the actuating plunger 4. In the process, the active ingredient is aspirated via the needle 14 and the interior of the hollow body 6 is thus filled.

After the syringe 1 has been made ready for use in one of the aforementioned ways and is accordingly in the state shown in FIGS. 1 and 8, the needle 14 is suitably positioned on the patient for the administration of the medical agent so that it pierces the patient's skin at a suitable point. The holding force of the needle 14 in the bearing sleeve 30 is thereby predetermined, in particular as described above by the suitable dimensioning of the components and/or the choice of the material pairing, such that the needle 14 remains securely in its position in the bearing sleeve 30 when the operator pierces the needle 14 through the patient's skin by handling it on the hollow body 6.

The actuating plunger 4 is then pressed by the operator so that the plunger or stopper 18 moves inside the hollow body 6 towards its distal end 8, thereby feeding the medical agent to the needle 14 and dispensing it via the latter. Shortly before complete dispensing of the active ingredient, i.e. shortly before complete emptying of the interior of the hollow body 6, the stopper 18 reaches the needle tip 42 of the needle 14 projecting into the interior in the vicinity of the distal end 8 of the hollow body 6, so that the latter penetrates into the receiving hole provided for this purpose in the needle bearing 62 of the gripper bar 54 as it moves further. The system thus again assumes the position already shown in FIG. 9, in which the proximal setting ring 58 strikes the surface 86 of the latching hook 84. Once again, the operator perceives this haptically.

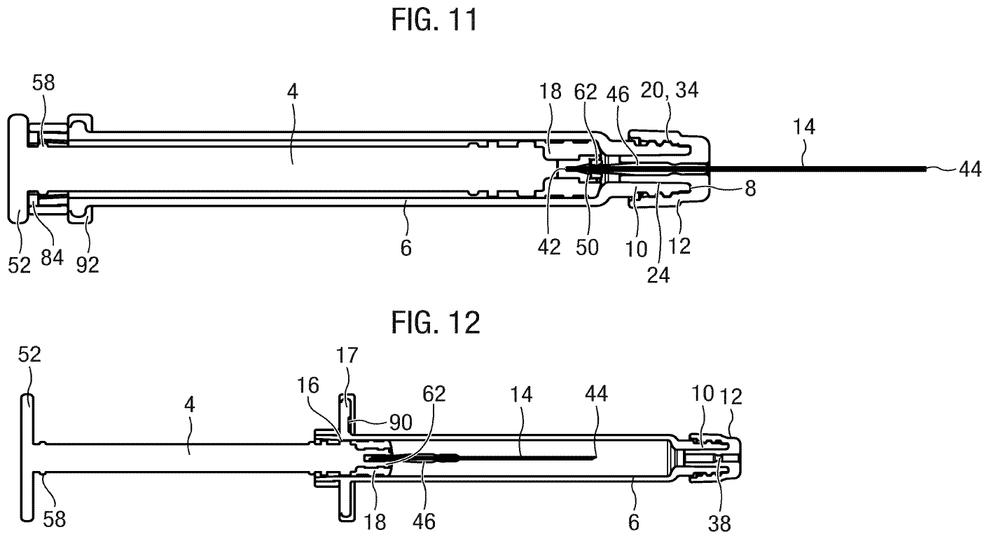

Unlike before, however, complete dispensing of the active substance is now provided. For this purpose, the operator applies a slightly increased force to further advance the stopper 18 in the hollow body 6, whereby the latching hooks 84 yield outwardly on the basis of suitable shaping and contouring on their surface, so that the adjusting ring 58 overcomes the aforementioned stop. Subsequently, as shown in the longitudinal section in FIG. 11, the piston 18 then reaches its end position (or also the “zero position”) directly at the distal end 8 of the hollow body 6. In this case, the needle bearing 62 encloses the end part of the hollow needle 14 projecting into the receptacle hole, whereby the latching lip running around the inside of the needle bearing 62 engages in the retaining groove formed by the latching edge 50 in the plastic jacket 46 of the needle overmold and thus fixes the needle 14 in the longitudinal direction. This insertion of the corresponding partial area of the hollow needle 14 into the needle bearing 62 and the connection of the needle 14 to the gripper bow 54 thereby achieved is also referred to herein as “connecting”.

In this context, the combination of the free space formed in the stopper 18 by the interaction with the design of the gripping bow 54, i.e. the said bypass, with the inner channel 24 in the needle holder 12, which widens outwardly in the manner of a cone, is considered to be independently inventive. Through this combination, even in the final phase during the administration of the active ingredient, a continuous, uniform delivery of the active ingredient can be made possible while minimizing any dead volume, so that even for the residual quantities of the active ingredient to be administered, a continuous injection and highly accurate dosing of the active ingredient can be ensured without having to change the feed force required for further movement of the actuating plunger 4.

After the active ingredient has been administered and in particular after the hollow body 6 has been completely emptied, the operator retracts the pusher plate 52 and with it the actuating plunger 4 as a whole. This also takes along the stopper 18 arranged at the end of the shaft 56 and pulls it within the hollow body 6 away from the distal end 8 towards the proximal end 16. In turn, it takes the needle 14, which is enclosed and fixed in the gripping bow 54, with it and pulls it into the hollow body 6, so that it is completely positioned inside the hollow body 6 in the final position. This position is shown in longitudinal section in FIG. 12. In this position, the needle 14 is safely stored inside the hollow body 6 and completely enclosed by it, so that there is no longer any danger from the needle 14 to personnel or operators, for example by accidental piercing. To fix this position, the second, distal adjustment ring 60 is provided, which is suitably latched to a latching element arranged on the hollow body 6. In this way, the flange-side adjusting ring 60 is securely anchored to the hollow body 6, and the needle 14 located in the hollow body 6 cannot be pulled further forwards or backwards, pushed or pushed.

In the mode of operation described, it is particularly important that, due to the suitable dimensioning of the components and/or a possible adhesive force due to the material pairing of the material of the plastic sheath 46 and the material of the bearing sleeve 30 surrounding it, the holding or break-away force of the needle 14 engaged in this way in the longitudinal direction is on the one hand sufficiently large, so that the needle 14 can be inserted into the patient's skin in accordance with the procedure described above, but on the other hand is also sufficiently small so that the described retraction movement of the needle 14 towards the interior of the hollow body 6 can be carried out.

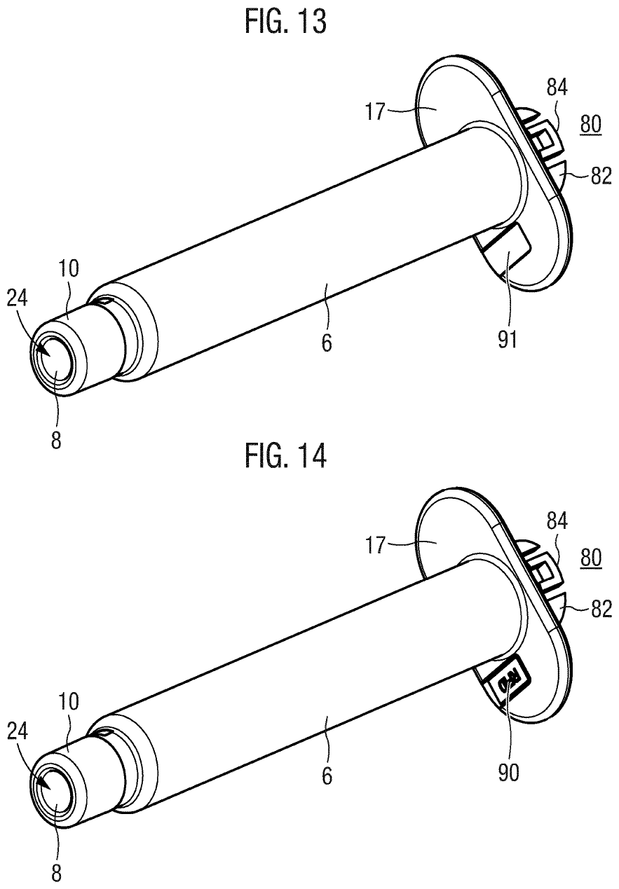

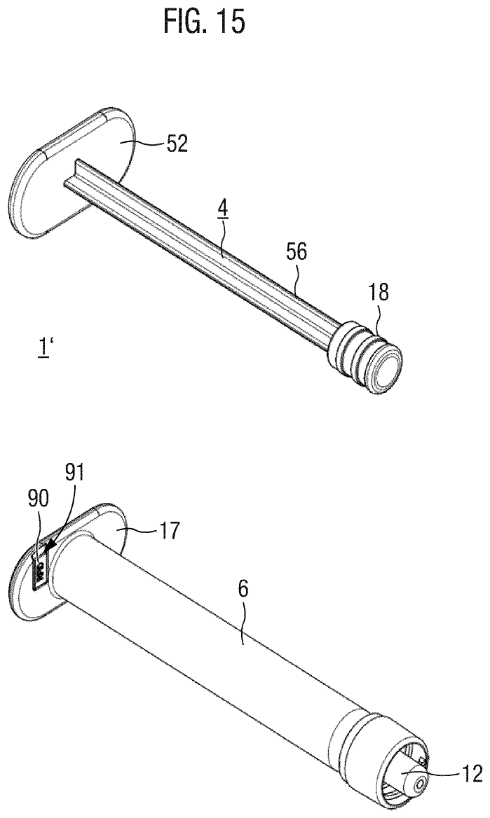

In a further embodiment considered to be independently inventive, the medical syringe 1, in particular for the purpose of setting up digitalized logistics, an automatable transport system for syringes, for quality or product control and/or for other recording or monitoring purposes, is equipped with an RFID chip 90, which can have, for example, an individualized, machine-readable coding. As can be seen from the representation of the syringe 1 in FIG. 1 and particularly clearly from the perspective representation of the hollow body 6 in FIGS. 13, 14, in this inventive embodiment the RFID chip 90 is positioned on the handle plate 17 formed on the hollow body 6. For this purpose, the grip plate is provided with a recess 91 for receiving the RFID chip 90 and additionally with a clipped-on finger rest 92, which on the one hand completely covers and thus protects the RFID chip 90 and on the other hand creates a pleasant feel for the user during operation. For this purpose, the finger pad 92 is made of PP.

Such equipment of a medical syringe 1′ with an RFID chip 90 in the area of its grip plate 17 is considered to be independently inventive. In accordance with this, FIG. 15 shows a medical syringe 1′ designed as a standard syringe with conventional actuating plunger 4 and conventional hollow body 6 forming the syringe housing, which is equipped with such an RFID chip 90 at its grip plate 17. This variant of the medical syringe 1′ can of course also be equipped with the protective cap 70 designed as a tamper-evident closure.

The medical syringe 1 described above is equipped with a retraction system as explained, in which the operator can completely retract the needle 14 into the hollow body 6 forming the syringe housing after complete delivery of the active ingredient. For this purpose, in this embodiment, after the aforementioned “connecting”, the actuating plunger 4 together with the stopper 18 arranged at the end and the needle bearing 62 enclosed by the latter, in which the needle 14 is fixed after the “connecting”, must be retracted manually or in the manner of an active action by the operator until the distal adjusting ring 60 is locked with the hollow body 6 in the manner described. This type of retraction system is therefore also referred to as “active retraction”.

In an alternative embodiment, a medical syringe 1″ may also be equipped with a “passive” retraction system suitable for “passive retraction”, as known in its basic mode of operation, for example, from EP 3 738 628 A1. The combination of such a passive retraction system with one or more of the features or groups of features described above, in particular with the design of the connection to the needle holder 12, with the equipment with RFID chip 90, with the equipment of the actuating plunger 4 with the adjusting rings 58, 60 as detent or stop elements and/or with the equipment with a protective cap 70 designed as a tamper-evident closure, is considered to be independently inventive.

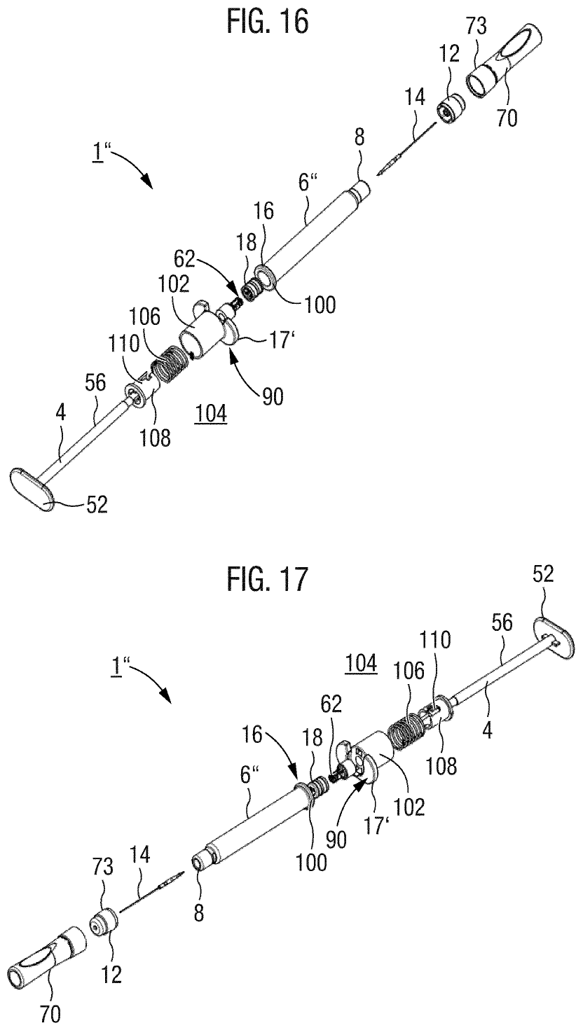

The components provided for forming such a passive retraction system of the medical syringe 1″ are shown in FIGS. 16, 17 in different viewing directions, each in exploded view. In this embodiment, the hollow body 6″ forming the syringe housing is also of an alternative design to that described above; it now comprises a separate grip plate 17′ which can be slid onto a latching edge 100 running around its proximal end 16. On this alternatively designed grip plate 17″, which incidentally in the embodiment example also carries the RFID chip 90 in accordance with the construction described above, the outer housing 102 of the retraction mechanism 104 is formed on its side facing away from the hollow body 6″. The medical syringe 1″, which is incidentally also provided with the tamper-evident closure 70 in a manner analogous to the construction described above, is provided with the desired passive needle protection by the retraction mechanism 104. The purpose of this is that after the syringe 1″ has been used, i.e. after the active ingredient held in the hollow body 6″ forming the syringe housing has been dispensed via the needle 14, the latter is drawn into the syringe housing in such a way that it is completely enclosed by the syringe housing. This is intended to keep unintentional contact with the used needle 14, for example by auxiliary or nursing personnel, and thus the risk of injury and contamination particularly low or, if possible, completely excluded.

Analogous to the design known from EP 3 738 628 A1, the retraction mechanism 104 is constructed according to the operating principle that a compressed spring 106 under pre-tension is locked by means of a locking element 108. The locking element 108 is unlocked at a given time as a result of the advance of the actuating plunger 4 and releases the spring 106 so that it can relax and thereby pushes back the actuating plunger 4.

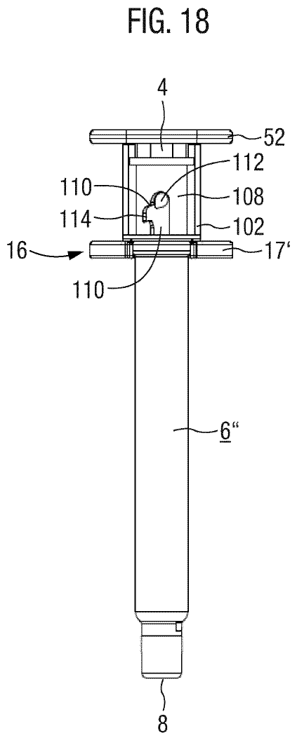

The design of the locking mechanism for the spring 106 is considered to be independently inventive. The locking mechanism is essentially based on the interaction of a number of control pins arranged on the inner surface of the outer housing 102, each with an associated guide slot 110 in the outer jacket of the locking element 108. As can be clearly seen in the side view of FIG. 18, the control pin 112 engages in the guide slot 110 formed in the outer jacket of the locking element 108. Laterally, the guide slot 110 has a bulge 114 into which the control pin 112 can be turned. The bulge 114 is contoured in such a way that the control pin 112 is held positively downwards, i.e. towards the hollow body 6″, and thus cannot move out of the way. In this state, in which the inner spring 106 is compressed, the system is thus locked and the spring 106 cannot relax.

Unlocking can then be effected by turning the control pin 112 laterally by suitable actuation and moving it out of the bulge 114. This brings it to lie over a downwardly open channel 116 of the guide link 110, so that it can now deflect downwardly relative to the locking element 108 and is no longer blocked in a form conclusive manner by the guide link 110. In this position, the locking element 108 can thus be displaced upwards relative to the outer housing 102, i.e. away from the hollow body 6″, without being blocked by the control pin 112 by the spring 106, whereby the spring 106 relaxes.

Thus, in response, i.e., to the release of the spring 106, the latter relaxes, thereby moving the pusher plate 52 of the actuating plunger 4 away from the syringe body formed by the hollow body 6″. This also entrains the needle bearing 62 connected to the shaft 56 of the actuating plunger 4, in which the needle 14 is already located at this stage, and pulls it within the hollow body 6″ away from the distal end 8 towards the proximal end 16. At the same time, it in turn takes the encased needle 14 with it and pulls it into the hollow body 6″ so that it is positioned completely inside the hollow body 6″ in the final state.

A significant and considered inventive aspect here is that the spring 106 provided for carrying out the retraction movement of the needle 14 into the hollow body 6″ is arranged above the grip plate 17′ in the region of the shaft 56 of the actuating plunger 4 and acts on the actuating plunger 4 via the plunger plate 52 thereof. In contrast to conventional retraction systems, in which the retraction spring is arranged in the region of the tip or the distal end of the syringe within the syringe body and acts directly on the needle or its bearing body, the design now provided according to the invention means that the needle 14 is moved during retraction together with the actuating plunger 4 and, in particular, its pusher plate 52, so that the user or operator can recognize the position and, above all, the change in position of the needle 14 on the basis of the position of the pusher plate 52 relative to the handle plate 17′. This recognition is possible visually, but above all also haptically.

In other words, the retraction movement generates a movement of the pusher plate 52 that can be felt by the user, for example with the thumb, similar to an actuation button of a ballpoint pen. Thus, the user can conclude from the movement of the pusher plate 52 that can be felt with the thumb, on the one hand, that the needle 14 has now been retracted and that there is therefore no longer any risk of injury or contamination in the region of the distal end 8 of the syringe 1″. On the other hand, however, it can also conclude from this that the active ingredient has now been completely administered without having to monitor this visually, for example. The syringe 1″ thus enables significantly simplified handling when administering the active ingredient.

### LIST OF REFERENCE SIGNS

1, 1′, 1″ Medical syringe
4 Actuating plunger
6, 6′, 6″ Hollow body
8 distal end
10 Connecting element
12 Needle holder
14 Hollow needle
16 Proximal end
17, 17′ Handle plate
18 Piston
20 External thread
22 Snap-in edge
24 Inner channel
30 Bearing sleeve
32 Contact surface
34 Female thread
36 Hexagon socket
38 Fixing lip
40 Needle tube
42, 44 Needlepoint
46 Plastic sheath
48 Holding groove
50 Snap-in edge
52 Handle plate
54 gripping yoke
56 Shank
58, 60 Adjusting ring
62 Needle bearing
64 Free space
70 Protective cap
72 Connecting bar
73 securing ring
74 Cap housing
76 Control panel
80 Snap-in mounting
82 Ring segments
84 Snap-in hook
86 Top
90 RF ID chip
91 Shaping
92 Finger rest
100 Latching edge
102 Outer casing
104 Retraction mechanism
106 Spring
108 Locking element
110 guide link
112 Control pin
114 Bulge
116 Channel

1. A hollow body for use as a syringe housing intended to receive a medical active substance, wherein a connecting element is integrally formed at a distal end of the hollow body, which has on the its inside an inner channel for connection to a needle holder for a hollow needle provided for injection of the medical active substance, which inner channel has an inner cross-section which widens continuously towards the distal end and which is provided on the its outside with means for mechanical fastening of the needle holder.

2. The hollow body of claim 1, wherein the inner channel is configured in the manner of a cone widening toward the distal end.

3. The hollow body of claim 2, wherein the cone formed by the internal channel has a cone angle of about 2°.

4. A medical syringe with a hollow body according to claim 1, which forms a syringe housing and is provided for receiving the medical active substance, and with a needle holder with a hollow needle provided for injecting the medical active substance, wherein the needle holder has a bearing sleeve for the hollow needle, which has on the its outside a conically designed contact surface adapted in its dimensioning, in particular with regard to its cross-sectional area, its diameter and/or its cone angle, to the inner channel of the connecting element and insertable therein.

5. The medical syringe of claim 4, having a hollow body which forms the syringe housing and is provided for accommodating a medical active ingredient and at whose proximal end a grip plate is integrally formed, on which an RFID chip is arranged.

6. The medical syringe of claim 4, having an actuating plunger and having a hollow body which forms the syringe housing and is provided for receiving a medical active ingredient, a number of stop and latching elements being arranged on a shaft of the actuating plunger.

7. The medical syringe of claim 6, the latching elements of which are designed as adjusting rings running around the shaft.
